(19) United States
(12) Patent Application Publication
(10) Pub. No.: US 2024/0307279 A1
(43) Pub. Date: Sep. 19, 2024

(54) FAST DRYING PERFUME COMPOSITION

(71) Applicant: Firmenich SA, Satigny (CH)

(72) Inventors: Philipp ERNI, Satigny (CH); Huda JERRI, Plainsboro, NJ (US); Rutger VAN SLEEUWEN, Plainsboro, NJ (US); Romane ANDRE, Satigny (CH); Lahoussine OUALI, Satigny (CH)

(21) Appl. No.: 18/575,618

(22) PCT Filed: Jun. 27, 2022
(86) PCT No.: PCT/EP22/67558

§ 371 Date: Dec. 29, 2023

Related U.S. Application Data
(60) Provisional application No. 63/216,533, filed on Jun. 30, 2021.

(30) Foreign Application Priority Data
Aug. 12, 2021 (EP) 21190985.8

(51) Int. Cl. A61K 8/34; A61Q 13/00; C11B 9/00

(52) U.S. Cl. CPC A61K 8/34; A61Q 13/00; C11B 9/00; A61K 2800/48

(57) ABSTRACT

Described herein is a fast-drying perfume composition including at least 70% of one or more fast evaporating solvents and less than 5% of one or more active ingredients. In some embodiments of the invention the solvent is selected from: ethanol, ethyl butyrate, ethyl acetate, isopropanol, 1-propanol, 1-pentanol, aromatic or aliphatic aldehydes with six or fewer C atoms, or fatty alcohols with six or fewer C atoms, 3-methoxy-3-methyl-1-butanol, amyl acetate, 1-pentanol, 2-methyl-1-butanol, 2-pentanol, 3-methyl-2-butanol, isoamyl alcohol, butyl acetate, butyl methyl ether, coconut alkanes, coco-caprylate/caprate, ethyl lactate, ethylene glycol mono n-propyl ether, 1-propanol, propylene glycol monoethyl ether, propylene glycol monomethyl ether acetate, propylene glycol monopropyl ether, lactones with a molecular weight below 80 g/mol, cyclotrisiloxane, cyclotetrasiloxane, cimethicone, octamethyltrisiloxane, and hexamethyldisiloxane. Also described herein are compositions, devices, and uses relating to the composition.

### INTRODUCTION

In recent years there has been a large increase in the use of face masks in society to reduce the risk of contracting infections caused by airborne pathogens. Typically such face masks are manufactured from several layers of paper or other non-woven materials, between which is a layer of filtering material to prevent the inhalation or exhalation of water droplets which can harbor such pathogens.

Face masks are prepared according to many different manufacturing standards, depending on the level or protection required, and in many different styles. Most are designed to be disposable after only a few times of use or after a period of time. Also, partly driven by the recent COVID pandemic, many users are opting to wear personalized or “designer” face masks in a form of fashion statement.

However, it can be appreciated over only a very short period of time the user may start to experience unwanted olfactory notes which are trapped against the mouth and nose. These olfactory notes arise from oral or body malodor from the user. This unpleasant effect of wearing face masks can discourage the public from adopting face mask usage which in times of high infection from airborne pathogens is clearly not desirable from a societal perspective.

Therefore the present inventors sought to develop a perfuming composition which could be readily adopted by a user to improve the user's experience while wearing a face mask, thereby improving the wellbeing of the user while wearing a face mask over extended periods of time

### SUMMARY OF THE INVENTION

An aspect of the invention provides a fast-drying perfume composition comprising at least 70% of one or more fast evaporating solvents and less than 5% of one or more active ingredients.

An embodiment of this aspect of the invention is wherein the fast evaporating solvent has vapor pressure of 100 Pa or more at a temperature of 25° C. Preferably the solvent is selected from: ethanol, ethyl butyrate, ethyl acetate, isopropanol, 1-propanol, 1-pentanol, aromatic or aliphatic aldehydes with six or fewer C atoms, or fatty alcohols with six or fewer C atoms, 3-methoxy-3-methyl-1-butanol, amyl acetate, 1-pentanol, 2-methyl-1-butanol, 2-pentanol, 3-methyl-2-butanol, isoamyl alcohol, butyl acetate, butyl methyl ether, coconut alkanes, coco-caprylate/caprate, ethyl lactate, ethylene glycol mono n-propyl ether, 1-propanol, propylene glycol monoethyl ether, propylene glycol monomethyl ether acetate, propylene glycol monopropyl ether, lactones with a molecular weight below 80 g/mol including propiolactones, cyclotrisiloxane, cyclotetrasiloxane, cimethicone, octamethyltrisiloxane, hexamethyldisiloxane.

A further embodiment of this aspect of the invention is wherein the perfume composition further comprises one or more of the following: water, a thickening agent and an emulsifying agent.

A further embodiment of this aspect of the invention is wherein the active ingredient comprises one or more of the following: perfuming ingredient, cooling agent, antimicrobial agent, antiviral agent, malodour counteracting agent.

A still further embodiment of the invention is wherein the composition comprises core shell microcapsules, a polymeric carrier matrix and/or spray-dried particles.

Another aspect of the invention provides a solid composition for applying a fragrance to a face mask, comprising the perfume composition of the invention and one or more of the following: a polymer, a fatty alcohol, a fatty acid, or salts thereof.

Another aspect of the invention provides a consumer product incorporating a perfume composition or solid composition of the invention

Another aspect of the invention provides a device for applying a incorporating a perfume composition or solid composition of the invention to a face mask. Preferably the device is spray bottle device or a solid stick dispenser device. In a further embodiment the device further comprises a matrix material suitable for stabilizing the perfume composition or solid composition during storage, to deliver the perfume composition or solid composition onto the mask using the applicator, and to release the fragrance during a certain time following application to the mask.

A further aspect of the invention provides a method to apply a fast-drying perfume composition to a face mask, the method comprising applying a perfume composition or solid composition of the invention.

A further aspect of the invention provides a method to manufacture a device for fragrancing a face mask comprising the steps of preparing blending, mixing and heating the components to form the solid composition of the invention, filling said solid composition into a dispenser device, and leaving the composition to solidify by letting it cool down below its temperature of solidification.

### DESCRIPTION OF THE FIGURES

FIG. 1: Comparison of scanning electron microscopy images of the different layers of the three-ply disposable mask (polypropylene) before and after application of two sprays on the inner face of the mask of different fragranced solutions containing 0.1% of limonene with different amounts of ethanol. Scale bars at the bottom right of each image correspond to 50 micrometers.

FIG. 2: Comparison of the liquid-repellent surface properties by measurement of the contact angle of a pure water drop on the different layers of the three-ply disposable mask (polypropylene) before and after application of two sprays on the inner face of the mask of different fragranced solutions containing 0.1% of limonene with different amount of ethanol.

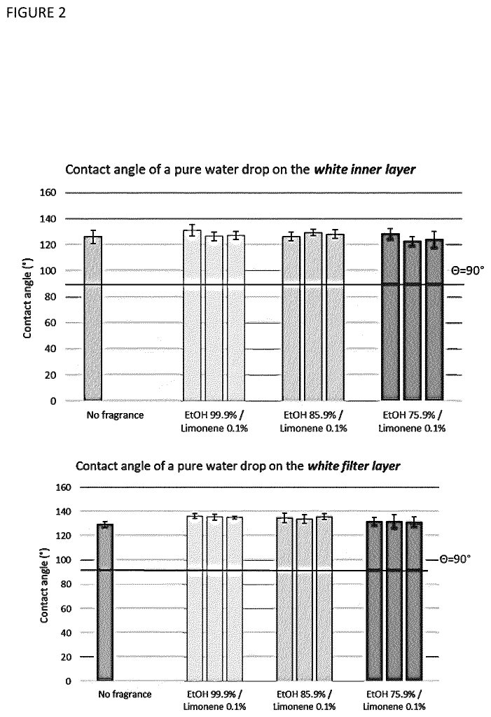

FIG. 3: Comparison of the surface properties by measurement of the contact angle of a pure water drop on the different layers of the 3-ply disposable mask (polypropylene) before and after application of ten sprays, respectively 5 on the inner face and 5 on the outer face of the mask of different fragranced solutions containing different fragrances with a constant amount of ethanol.

FIG. 4: Comparison of the surface properties by measurement of the contact angle of a pure water drop on the different layers of the cloth mask Enerplex before and after application of ten sprays, respectively 5 on the inner face and 5 on the outer face of the mask of a fragranced solution containing a complex fragrances with a constant amount of ethanol. These ten sprays mimic a daily usage of the sprays (two sprays every 2 h so ten sprays per day (10 h)). Each value is an average of 10 measurements performed on the same piece of mask. Important damage has been noticed for the inner and middle layer made of polyester and cotton where there is a complete loss of surface properties (i.e. water repellency) after the application of the product.

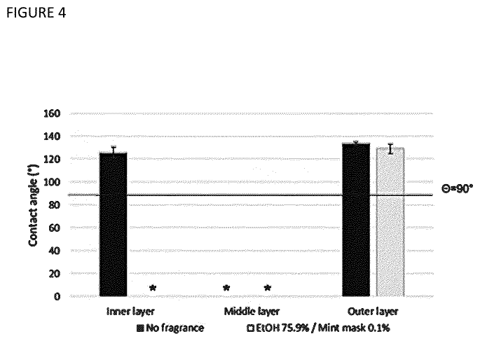

FIG. 5: Comparison of the surface properties by measurement of the contact angle of a pure water drop on the different layers of a cloth mask before and after application of ten sprays, respectively 5 on the inner face and 5 on the outer face of the mask of a fragranced solution containing a complex fragrance composition with a constant amount of ethanol. These ten sprays mimic a daily usage of the sprays (two sprays every 2 h so ten sprays per day (10 h)). Each value is an average of 10 measurements performed on the same piece of mask. No significant changes noticed before and after the application of the fragrance. The fabric layer made of cotton and polyester has no water repellency properties. Left column shown for each layer: water drop on a mask layer without any fragrance applied beforehand; right column shown for each layer: water drop on a mask layer measured after applying the fragrance composition and drying.

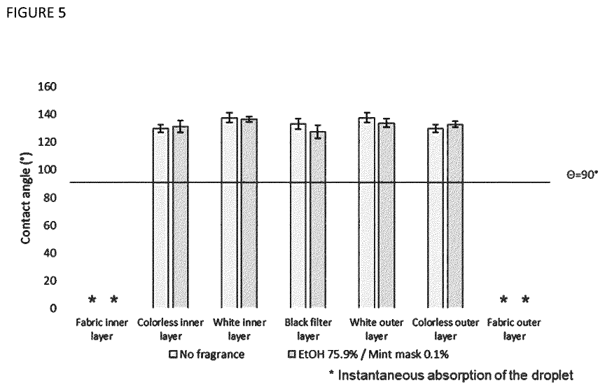

FIG. 6: Comparison of scanning electron microscopy images of the different layers of the three-ply disposable mask (polypropylene) before and after soaking in ethanol or limonene. For the case of limonene, a deposit is visible as white layer in the micrograph, indicating an undesired modification of the mask microstructure, pointed out in the image by an explanation mark. The untreated mask reference and a mask treated with pure ethanol are shown as comparison for intact, unchanged microstructure. The scale bar at the bottom right in each image corresponds to 50 micrometers.

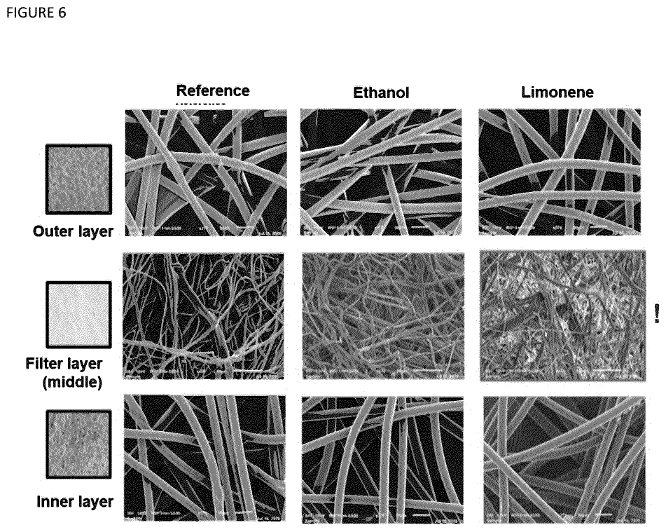

FIG. 7: Comparison of the surface properties by measuring the contact angle of a pure water drop on the different layers of a three-ply disposable mask (polypropylene) before and after soaking the mask in ethanol or limonene. The only significant change is the loss of surface properties (i.e. water repellency) after soaking the white filter layer in the limonene.

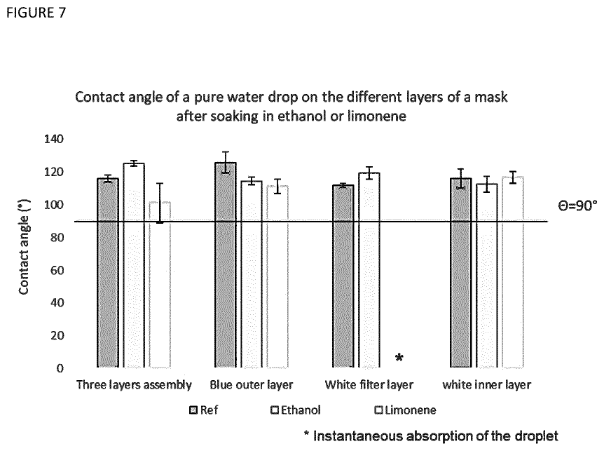

### DETAILED DESCRIPTION

As explained above, the present inventors sought to prepare a perfume composition which could be used for application to face masks, preferably disposable face masks. When preparing the composition, they determined that two characteristics were particularly important.

Firstly, the solvent used in the perfume composition should be “fast evaporating” so that when applied to the face mask it could be used in a short period of time. Moreover, a “fast evaporating” solvent would reduce the risk of any adverse reactions with the structural materials of the mask.

Secondly, since the face mask is by design intended to be worn in close proximity to the nose and mouth of the user, the perfume composition should not contain a large amount of perfuming ingredient.

From this realization, the inventors prepared a fast evaporating perfume composition of the invention.

Hence aspect of the invention provides a fast-drying perfume composition comprising at least 70% of one or more fast evaporating solvents and less than 5% of one or more active ingredients.

The invention solves the problem of poor olfactory user experience due to oral and/or body malodour while wearing face masks, thereby also allowing to enhance mask wearer's compliance with sanitary measures such as face mask mandates.

The results observed using the invention are an improved olfactory experience for the mask user and facilitated compliance with sanitary measures such as face mask mandates.

By “fast drying” we mean that when applied to a surface, 0.5 gram of the composition deposited on 1 square meter of mask tissue will evaporate leaving only the active ingredient in less than or equal to one minute. Preferably the composition dries in less than 45 seconds, preferably less than 30 seconds or less.

As can be appreciated such a fast evaporation time is of great benefit for the user of the face mask since it reduces the time needed for the mask to suitable for use, and hence aids the consumer experience.

The fast-drying perfume composition of the invention comprises at least 70% of a fast evaporating solvent, preferably at least 75%, 80%, 85%, 86%, 87%, 88%, 89%, 90%, 91%, 92%, 93%, 94%, 95%, 96%, 97%, 98%, 99%, 99.5% or more.

By “fast evaporating” we mean that the solvent has a vapor pressure of 100 Pa or more at a temperature of 25° C.

Examples of solvents which can be used in the perfume composition of the invention include ethanol, ethyl butyrate, ethyl acetate, isopropanol, 1-propanol, 1-pentanol, aromatic or aliphatic aldehydes with six or fewer C atoms, or fatty alcohols with six or fewer C atoms, 3-methoxy-3-methyl-1-butanol, amyl acetate, 1-pentanol, 2-methyl-1-butanol, 2-pentanol, 3-methyl-2-butanol, isoamyl alcohol, butyl acetate, butyl methyl ether, coconut alkanes, coco-caprylate/caprate, ethyl lactate, ethylene glycol mono n-propyl ether, 1-propanol, propylene glycol monoethyl ether, propylene glycol monomethyl ether acetate, propylene glycol monopropyl ether, lactones with a molecular weight below 80 g/mol including propiolactones, cyclotrisiloxane, cyclotetrasiloxane, cimethicone, octamethyltrisiloxane, hexamethyldisiloxane.

The fast-drying perfume composition of the invention comprises less than 5% of an active ingredient, preferably less than 4%, 3%, 2.5%, 2%, 1.5%, 1%, 0.9%, 0.8%, 0.7%, 0.6%, 0.5%, 0.4%, 0.3%, 0.2%, 0.1% or less.

By “active ingredient” we include one or more of the following: a perfuming ingredient, cooling agent, antimicrobial agent, antiviral agent, malodour counteracting agent.

By “perfuming ingredients” it is understood here compounds which are used as active ingredients in perfuming preparations or compositions in order to impart a hedonic effect when applied to a surface. In other words, such compounds, to be considered as being perfuming ones, must be recognized by a person skilled in the art of perfumery as being able to impart or modify in a positive or pleasant way the odor of a composition or of an article or surface, and not just as having an odor. Moreover, this definition is also meant to include compounds that do not necessarily have an odor but are capable of modulating the odor of a perfuming composition, perfumed article or surface and, as a result, of modifying the perception by a user of the odor of such a composition, article or surface. It also contains malodor counteracting ingredients and compositions.

By “cooling agents” we include, for example, those set forth in U.S. Pat. Nos. 9,394,287 and 9,732,071. Additional examples of cooling agents include 5-methyl-2-(propane-2-yl)cyclohexyl-N-ethyloxamate, N-ethyl-p-menthane carboxamide (WS-3, also referred to as menthane-3-carboxylic acid-N-ethyl amide), N-2,3-trimethyl-2-isopropyl butane amide (WS-23), menthyl lactate (Frescolat.RTM. ML), menthone glycerine acetal (Frescolat.RTM. MGA), mono-menthyl succinate (Physcool.RTM.), mono-menthyl glutarate, O-menthyl-glycerine, menthyl-N, N-dimethyl succinamate, N-(4-cyano methyl phenyl)-p-menthane carboxamide, N-(2-(pyridin-2-yl)ethyl)-3-p-menthane carboxamide, menthol and menthol derivatives (e.g. L-menthol, D-menthol, racemic menthol, isomenthol, neoisomenthol, neomenthol) menthyl ether (e.g. (I-menthoxy)-1,2-propanediol, (I-menthoxy)-2-methyl-1,2-propanediol, 1-menthyl-methyl ether), menthyl ester (e.g. menthyl formiate, menthyl acetate, menthyl isobutyrate, menthyl lactates, L-menthyl-L-lactate, L-menthyl-D-lactate, menthyl-(2-methoxy)acetate, menthyl-(2-methoxy ethoxy)acetate, menthyl pyroglutamate), N-(4-cyano methyl phenyl)-p-menthane carboxamide, N-(2-(pyridin-2-yl)ethyl)-3-p-menthane carboxamides, menthyl carbonates (e.g. menthyl propylene glycol carbonate, menthyl ethylene glycol carbonate, menthyl glycerine carbonate or mixtures thereof), menthane carboxylic acid amide (e.g. menthane carboxylic acid-N-ethylamid [WS3], N-alpha.-(menthane-carbonyl)glycine ethyl ester [WS5], menthane carboxylic acid-N-(4-cyanophenyl)amide, menthane carboxylic acid-N-(alkoxyalkyl)amide), menthone and menthone derivatives (e.g. L-menthone glycerine ketal), 2,3-dimethyl-2-(2-propyl)-butyric acid derivatives (e.g. 2,3-dimethyl-2-(2-propyl)-butyric acid-N-methyl amide [WS23]), isopulegol or its esters (1-(−)-isopulegol, 1-(−)-isopulegol acetate), menthane derivatives (e.g. p-menthane-3,8-diol), N-(4-cyano methyl phenyl)-p-menthane carboxamides, N-(2-(pyridin-2-yl)ethyl)-3-p-menthane carboxamides, cubebol or synthetic or natural mixtures containing cubebol, pyrrolidone derivates of cycloalkyl dione derivatives (e.g. 3-methyl-2(1-pyrrolidinyl)-2-cyclopentene-1-one) or tetrahydropyrimidine-2-ones (e.g. Icilin or related compounds such as those described in WO 2004/026840), N-(4-cyano methyl phenyl)-p-menthane carboxamide, N-(2-(pyridin-2-yl)ethyl)-3-p-menthane carboxamides, menthyl ether (e.g. (I-menthoxy)-1,2-propanediol, (I-menthoxy)-2-methyl-1,2-propanediol), more polar menthyl esters (e.g. menthyl lactates, L-menthyl-L-lactate, L-menthyl-D-lactate, menthyl-(2-methoxy)acetate, menthyl-(2-methoxy ethoxy)acetate, menthyl pyroglutamate), menthyl carbonates (e.g. menthyl propylene glycol carbonate, menthyl ethylene glycol carbonate, menthyl glycerine carbonate), the semi-esters of menthols with a dicarboxylic acid or the derivatives thereof (e.g. mono-menthyl succinate, mono-menthyl glutarate, mono-menthyl malonate, O-menthyl succinic acid ester-N, N-(dimethyl)amide, O-menthyl succinic acid ester amide), 3,4-methylendioxycinnamic acid-N-cyclohexyl-N-2-pyridylamide, isopropyl-(5-methoxy-2-pyridin-2-yl-pyrimidin-4-yl)-amine, 3,4,6,7,11b, 12-hexahydro-3,3-dimethyl-spiro[13H-dibenzo[a,f]quinolizine-1--3,2′-[1,3]dithiolan]-1 (2H)-one, 5,6,10b, 11-tetrahydro-3-methyl-spiro[12H-benzo[a]furo[3,4-f]quinolizine-1--2,2′-[1,3]dithiolan]-1(3H)-one. Most preferred as cooling compounds are compounds selected from the group consisting of 5-methyl-2-(propane-2-yl)cyclohexyl-N-ethyloxamate, N-ethyl-p-menthane carboxamide (WS-3, also referred to as menthane-3-carboxylic acid-N-ethyl amide), menthyl lactate (Frescolat.RTM. ML), menthone glycerine acetal (Frescolat.RTM. MGA), N-(4-cyano methyl phenyl)-p-menthane carboxamide and (I-menthoxy)-1,2-propanediol, 2-(4-methylphenoxy)-N-(1H-pyrazol-5-yl)-N-(2-thienylmethyl)acetamide, 2-(4-methylphenoxy)-N-(1H-pyrazol-3-yl)-N-(2-thienylmethyl)acetamide and a mixture of 2-(4-methylphenoxy)-N-(1H-pyrazol-5-yl)-N-(2-thienylmethyl)acetamide and 2-(4-methylphenoxy)-N-(1H-pyrazol-3-yl)-N-(2-thienylmethyl)acetamide.

By “antimicrobial agent” we include cetyl-trimethylammonium bromide, cetyl pyridinium chloride, benzethonium chloride, diisobutyl phenoxy ethoxy ethyl dimethyl benzyl ammonium chloride, sodium N-lauryl sarcosine, sodium N-palmethyl sarcosine, lauroyl sarcosine, N-myristoyl glycine, potassium N-lauryl sarcosine, trimethyl ammonium chloride, sodium aluminum chlorohydroxy lactate, triethyl citrate, tricetylmethyl ammonium chloride, 2,4,4′-trichloro-2′hydroxy diphenyl ether (triclosan), 3,4,4′-trichlorocarbanilide (triclocarban), diaminoalkyl amides such as L-lysine hexadecyl amide, heavy metal salts of citrate, salicylate, and piroctose, especially zinc salts, and acids thereof, heavy metal salts of pyrithione, especially zinc pyrithione, zinc phenolsulfate, famesol, and combinations thereof.

By “antiviral agents” we include agents well known in the art to the skilled person. for example, monoterpene hydrocarbons contained in plant extracts such as osimene, camphenol, limonene, sabinen, milsen, terpinen, pinen, and simen, and monoterpene hydrocarbons such as citronellol, geraniol, isopregol, linalol, and terpinerol. Phenols such as terpene alcohols, anetol, carbachlor, eugenol, timol, paracresol, and cabicol, phenol ethers such as t-annetol, chabicol methyl ether, and saflor, acetaldehyde, citral, citroneral, geranial, perylaldehyde, valeranal Aldehydes such as acetophenone, camphor, jasmon, notecaton, menthon, phenol, carboxylic, pregon and other ketones, caliolefin oxide, cineol, bisabolol oxide and other oxides, catechin, proanthocyanidin, flavon, etc. One or several kinds can be appropriately selected and used from the medicinal ingredients such as flavanone, anthocyanin, phenols, flavonoids such as flavonol, and the like. In addition to the above, for example, organic acids such as ascorbic acid, carboxylic acid, and citric acid, anionic surfactants, cationic surfactants, amphoteric surfactants, nonionic surfactants, and the like can also be used. it can.

By the term “malodor counteracting ingredient” we mean here compounds which are capable of reducing the perception of malodor, i.e. of an odor that is unpleasant or offensive to the human nose by counteracting and/or masking malodors. In a particular embodiment, these compounds have the ability to react with key compounds causing known malodors. The reactions result in reduction of the malodor materials' airborne levels and consequent reduction in the perception of the malodor. Such agents are known in the art to the skilled person. For example, such agents can include aroma and/or non-aroma chemicals which are known to have an action in reducing the perception of the intensity of malodours e.g. unsaturated esters, ketones, aldehydes, and/or a fragrant material e.g. citronellal and/or cedarwood oil.

In a preferred aspect of the invention, the active ingredient is selected from flavours and fragrances.

For the purpose of the present invention, the terms “flavour or fragrance” encompass flavour or fragrance ingredients or compositions of current use in the flavour and/or fragrance industry, of both natural and synthetic origin. It includes single compounds and mixtures. Specific examples of such flavour or fragrance ingredients may be found in the current literature, e.g. in Fenaroli's Handbook of flavour ingredients, 1975, CRC Press; Synthetic Food adjuncts, 1947 by M. B. Jacobs, edited by Van Nostrand; or Perfume and Flavor Chemicals by S. Arctander, 1969, Montclair, New Jersey (USA). Many other examples of current flavouring and/or perfuming ingredients may be found in the patent and general literature available. The flavouring or perfuming ingredients may be present in the form of a mixture with solvents, adjuvants, additives and/or other components, generally those of current use in the flavours and fragrance industry.

The perfume composition of any the invention may further comprising one or more of the following: water, a thickening agent and an emulsifying agent.

By “thickening agent” we include polyacrylate cross-polymer, organosilicones, modified castor oil, polyvinyl alcohols, cellulose derivative, hydroxypropyl methyl cellulose, starch derivative, carrageenans, pectins, agar, carob gum, guar gum, konjac gum, mesquite gum, apple fiber, citrus fiber, potato fiber, pea fiber, and maltodextrins.

By “emulisifing agent” we include lecithin, glycerol esters, fatty acid esters, saponins, proteins, gum Arabic, hydrophobically modified starches, sorbitan esters, polysorbates and mixtures thereof.

In a preferred embodiment of the invention the composition is formulated as core shell microcapsule, a polymeric carrier matrix and/or is in the form of a spray-dried particles.

By carrier or carrier material is herein understood that the material of the carrier is suitable to entrap, encapsulate or hold a certain amount of fast drying perfume composition of the invention. In order to be qualified as a carrier material, the carrier material has to entrap, encapsulate or hold a certain amount of perfume composition.

Typically, when the fast drying perfume composition of the invention is in a matrix form, the carrier material is a matrix material.

Typically, when the fast drying perfume composition of the invention is in the form a core-shell microcapsule, the carrier is a shell and entraps preferably at least 80 wt. %, preferably at least 90 wt. %, of the fast drying perfume composition of the invention, based on the total weight of the delivery system.

In a particular embodiment, the carrier or carrier material is a solid carrier material, i.e. an emulsion or solvent is not a carrier or carrier material.

In a particular embodiment, the fast drying perfume composition of the invention is a core-shell microcapsule or the delivery system is in a matrix form (i.e fast drying perfume composition entrapped within a polymeric matrix, for example a monomeric, oligomeric or polymeric carrier matrix). For the sake of clarity, thereby it is understood that when the fast drying perfume composition of the invention is a core-shell microcapsule, the fast drying perfume composition of the invention is comprised in the core which is surrounded or entrapped by the shell. When the fast drying perfume composition of the invention is in the form of a matrix, the fast drying perfume composition of the invention is entrapped in a matrix of a carrier, such as a monomeric, oligomeric or polymeric carrier matrix, by adsorption in the matrix.

In case the carrier is a monomeric, oligomeric or polymeric carrier matrix, it is herein understood that the fast drying perfume composition of the invention is entrapped in the monomeric, oligomeric or polymeric carrier matrix by adsorption within the monomeric, oligomeric or polymeric carrier matrix, i.e. it is adsorbed in the pores of the monomeric, oligomeric or polymeric carrier matrix.

In a particular embodiment, the carrier material comprises a monomeric, oligomeric or polymeric carrier material, or mixtures of two or more of these. An oligomeric carrier is a carrier wherein 2-10 monomeric units are linked by covalent bonds. For example, if the oligomeric carrier is a carbohydrate, the oligomeric carrier may be sucrose, lactose, raffinose, maltose, trehalose, fructo-oligosaccharides.

Examples of a monomeric carrier materials are glucose, fructose, mannose, galactose, arabinose, fucose, sorbitol, mannitol, for example.

Polymeric carriers have more than 10 monomeric units that are linked by covalent bonds.

In a particular embodiment, the carrier may be a polymeric carrier material. Non-limiting examples of polymeric carrier material includes polyaspartate, modified polysuccinimides, lignin and its derivatives, polyoxazoline, polyhydroxyalcanoates, polyphenols, natural and synthetic clays, polyvinyl acetates, polyvinyl alcohol, dextrines, maltodextrines, glucose syrups, natural or modified starch, polysaccharides, carbohydrates, chitosan, gum Arabic, polyethylene glycol, polyvinyl pyrrolidone, polyvinyl alcohol, acrylamides, acrylates, polyacrylic acid and related, maleic anhydride copolymers, amine-functional polymers, vinyl ethers, styrenes, polystyrenesulfonates, vinyl acids, ethylene glycol-propylene glycol block copolymers, vegetable gums, gum acacia, pectins, xanthanes, alginates, carragenans or cellulose derivatives, such as carboxymethyl methylcellulose, methylcellulose or hydroxyethyl cellulose; chitin, proteins (animal and vegetal), polyaspartate, poylsuccinimides and its derivatives, polyesters, polyaminoesters, polyhydroxyalkanoates, polycarbonates and mixture thereof. Preferably the polymeric carrier material comprises natural or modified starch, maltodextrins, carbohydrates, chitin, proteins (animal and vegetal), polyaspartate, poylsuccinimides and its derivatives, polyesters, polyaminoesters, polyhydroxyalkanoates, polycarbonates and mixtures thereof.

The carrier material is preferably present in an amount between 25 and 80 wt. %, preferably between 30 and 60 wt. % and more preferably between 40 and 55 wt. % (based on the total weight of the delivery system).

A still further embodiment of the invention is wherein the composition comprises core shell microcapsules, a polymeric carrier matrix and/or spray-dried particles.

A particularly preferred embodiment of the invention is wherein the composition is a suitable for a roll-on application device and comprises 71.75% ethanol, 25% water, 0.25% pPolyacrylate crosspolymer 6, and 3% of the fast drying composition of the invention. Preferably the fast drying composition of the invention incorporates a mint fragrance.

A further particularly preferred embodiment of the invention is wherein the composition is a suitable for a roll-on application device and comprises 70% ethanol, 25% water, 2% hydroxypropyl methylcellulose (HPMC), and 3% of the fast drying composition of the invention. Preferably the fast drying composition of the invention incorporates a mint fragrance.

Another aspect of the invention provides a solid composition for applying a fragrance to a face mask, comprising the perfume composition of the invention and one or more of the following: a polymer, a fatty alcohol, a fatty acid, or salts thereof

A particularly preferred embodiment of this aspect of the invention is wherein the solid composition comprises 72% propylene glycol, 17% Stearyl alcohol, 8% Stearate sodium, and 3% of the fast drying composition of the invention. Preferably the fast drying composition of the invention incorporates a mint fragrance.

Another aspect of the invention provides a consumer product incorporating a perfume composition or solid composition of the invention

Another aspect of the invention provides a device for applying a incorporating a perfume composition or solid composition of the invention to a face mask. Preferably the device is spray bottle device or a solid stick dispenser device. In a further embodiment the device further comprises a matrix material suitable for stabilizing the perfume composition or solid composition during storage, to deliver the perfume composition or solid composition onto the mask using the applicator, and to release the fragrance during a certain time following application to the mask.

A further aspect of the invention provides a method to apply a fast-drying perfume composition to a face mask, the method comprising applying a perfume composition or solid composition of the invention.

A further aspect of the invention provides a method to manufacture a device for fragrancing a face mask comprising the steps of preparing blending, mixing and heating the components to form the solid composition of the invention, filling said solid composition into a dispenser device, and leaving the composition to solidify by letting it cool down below its temperature of solidification.

The compositions presented herein may be incorporated into any consumer products and devices for applying the fast drying perfume compositions of the invention. Exemplary products include wax-based sticks, soap-based sticks, compressed powder sticks, roll-on suspensions or solutions, emulsions, gels, creams, squeeze sprays, pump sprays, aerosols, and the like. Each product form may contain its own selection of additional components, some essential and some optional. The types of components typical for each of the above product forms may be incorporated in the corresponding compositions presented herein.

Depending of the type of product, the consumer products and devices may comprise supplementary ingredients enabling to obtain the desired form. Non-limiting examples of suitable ingredients include emollient(s), solubilizer(s), antioxidant(s), preservative(s), carrier(s), odour entrapper(s), propellant(s), primary structurant(s), additional chassis ingredient(s), volatile silicone solvent(s), gellant(s), buffering agent and residue masking material(s). A person skilled in the art is able to select them on the basis of its general knowledge and according to intended form of the consumer products and devices.

For example, by way of illustration, a roll-on product may comprise water, emollient, solubilizer, antioxidants, preservatives, or combinations thereof; a clear gel product may comprise water, emollient, solubilizer, antioxidants, preservatives, ethanol, or combinations thereof; a spray product may contain a carrier, odour entrappers, propellant, or combinations thereof; an solid product may contain a primary structurant, and additional chassis ingredient(s); a soft product may comprise volatile silicone, gellant, residue masking material, or combinations thereof; an aerosol product may comprise a carrier, a propellant, or a combination thereof.

Emollients suitable for products include, but are not limited to, propylene glycol, polypropylene glycol (like dipropylene glycol, tripropylene glycol, etc.), diethylene glycol, triethylene glycol, neopentyl glycol diheptanoate, PEG-4, PEG-8, 1,2-pentanediol, 1,2-hexanediol, hexylene glycol, glycerin, C2 to C20 monohydric alcohols, C2 to C40 dihydric or polyhydric alcohols, alkyl ethers of polyhydric and monohydric alcohols, dicaprylyl carbonate, dicaprylyl ether, diethylhexylcyclohexane, dibutyl adipate, volatile silicone emollients such as cyclopentasiloxane, nonvolatile silicone emollients such as dimethicone, mineral oils, polydecenes, petrolatum, and combinations thereof. One example of a suitable emollient comprises PPG-15 stearyl ether. Other examples of suitable emollients include dipropylene glycol and propylene glycol.

Antimicrobial agents may comprise cetyl-trimethylammonium bromide, cetyl pyridinium chloride, benzethonium chloride, diisobutyl phenoxy ethoxy ethyl dimethyl benzyl ammonium chloride, sodium N-lauryl sarcosine, sodium N-palmethyl sarcosine, lauroyl sarcosine, N-myristoyl glycine, potassium N-lauryl sarcosine, trimethyl ammonium chloride, sodium aluminum chlorohydroxy lactate, triethyl citrate, tricetylmethyl ammonium chloride, 2,4,4′-trichloro-2′hydroxy diphenyl ether (triclosan), 3,4,4′-trichlorocarbanilide (triclocarban), diaminoalkyl amides such as L-lysine hexadecyl amide, heavy metal salts of citrate, salicylate, and piroctose, especially zinc salts, and acids thereof, heavy metal salts of pyrithione, especially zinc pyrithione, zinc phenolsulfate, famesol, and combinations thereof.

Suitable odour entrappers for use herein include, for example, solubilized, water-soluble, uncomplexed cyclodextrin. As used herein, the term “cyclodextrin” includes any of the known cyclodextrins such as unsubstituted cyclodextrins containing from six to twelve glucose units, including alpha-cyclodextrin, beta-cyclodextrin, gamma-cyclodextrin and/or their derivatives and/or mixtures thereof.

Alternative malodour entrappers can be zinc ricinoleate & derivatives such as TEGO® SORB B 80, TEGO® Sorb Cone. 50& TEGO® SORB A 30.

A suitable solubilizer can be, for example, a surfactant, such as a no-foaming or low-foaming surfactant. Suitable surfactants are nonionic surfactants, cationic surfactants, amphoteric surfactants, zwitterionic surfactants, and mixtures thereof. Suitable solubilizers include, for example, polyethylene glycol ether of cetearyl alcohol, hydrogenated castor oil such as polyoxyethylene hydrogenated castor oil, polyoxyethylene 2 stearyl ether, polyoxyethylene 20 stearyl ether, and combinations thereof.

Suitable preservatives include organic sulfur compounds, halogenated compounds, cyclic organic nitrogen compounds, low molecular weight aldehydes, parabens, propane diol materials, isothiazolinones, quaternary compounds, benzoates, low molecular weight alcohols, dehydroacetic acid, phenyl and phenoxy compounds, or mixtures thereof.

Non-limiting examples of commercially available preservatives include a mixture of about 77%5-chloro-2-methyl-4-isothiazolin-3-one and about 23%2-methyl-4-isothiazolin-3-one, a broad spectrum preservative available as a 1.5% aqueous solution under the trade name Kathan® CG by Rohm and Haas Co.; 5-bromo-5-nitro-1,3-dioxane, available under the tradename Bronidox L® from Henkel; 2-bromo-2-nitropropane-1,3-diol, available under the trade name Bronopol® from Inolex; I,G-hexamethylene bis(5-(p-chlorophenyl)biguanide), commonly known as chlorhexidine, and its salts, e.g., with acetic and digluconic acids; a 95:5 mixture of 1,3-bis(hydroxymethyl)-5,5-dimethyl-2,4-imidazolidinedione and 3-butyl-2-iodopropynyl carbamate, available under the trade name Glydant Plus® from Lonza; N-[1,3-bis(hydroxymethyl)2,5-dioxo-4-imidazolidinyl]-N, N′-bis(hydroxy-methyl) urea, commonly known as diazolidinyl urea, available under the trade name Germall® II from Sutton Laboratories, Inc.; N,N″-methylenebis {N′-[I-(hydroxymethyl)-2,5-dioxo-4-imidazolidinyl]urea}, commonly known as imidazolidinyl urea, available, e.g., under the trade name Abiol® from 3 V-Sigma, Unicide U-13® from Induchem, German 115® from Sutton Laboratories, Inc.; polymethoxy bicyclic oxazolidine, available under the trade name Nuosept® C from Hills America; formaldehyde; glutaraldehyde; polyaminopropyl biguanide, available under the trade name Cosmocil CQ® from ICI Americas, Inc., or under the trade nameMikrokill® from Brooks, Inc; dehydroacetic acid; and benzsiothiazolinone available under the trade name Koralone™ B-I 19 from Rohm and Hass Corporation.

Suitable levels of preservative can range from about 0.0001% to about 0.5%, alternatively from about 0.0002% to about 0.2%, alternatively from about 0.0003% to about 0.1%, by weight of the composition.

Suitable carriers can include, water, alcohol, or combinations thereof. Useful alcohols include C₁-C₃ alcohols. In some aspects, the alcohol is ethanol.

Some examples of propellants include compressed air, nitrogen, inert gases, carbon dioxide, and mixtures thereof. Propellants may also include gaseous hydrocarbons like propane, n-butane, isobutene, cyclopropane, and mixtures thereof; e.g. A-46 (a mixture of isobutane, butane and propane), A-31 (isobutane), A-17 (n-butane), A-108 (propane), AP70 (a mixture of propane, isobutane and nbutane), AP40 (a mixture of propane, isobutene and n-butane), AP30 (a mixture of propane, isobutane and n-butane). Some non-limiting examples of propellants include 1,1-difluoroethane, 1,1,1,2,2-pentafluoroethane, 1,1,1,2-tetrafluoroethane, 1,1,1,2,3,3,3-heptafluoropropane, trans-1,3,3,3-tetrafluoroprop-I-ene, dimethyl ether, dichlorodifluoromethane (propellant 12), I,I-dichloro-1,1,2,2-tetrafluoroethane (propellant 114), I-chloro-I,I-difluoro-2,2-trifluoroethane (propellant 115), I-chloro-I,I-difluoroethylene (propellant 142B), 1,1-difluoroethane (propellant 152A), monochlorodifluoromethane, and mixtures thereof.

The term “primary stmcturant” as used herein means any material known or otherwise effective in providing suspending, gelling, viscosifying, solidifying, and/or thickening properties to the composition or which otherwise provide structure to the final product form. These primary structurants include gelling agents, and polymeric or non-polymeric or inorganic thickening or viscosifying agents. Such materials will typically be solids under ambient conditions and include organic solids, crystalline or other gellants, inorganic particulates such as clays or silicas, or combinations thereof. Non-limiting examples of suitable primary structurants include stearyl alcohol and other fatty alcohols; hydrogenated castor wax (e.g., Castorwax MP80, Castor Wax, etc.); hydrocarbon waxes include paraffin wax, beeswax, camauba, candelilla, spermaceti wax, ozokerite, ceresin, baysberry, synthetic waxes such as Fischer-Tropsch waxes, and microcrystalline wax; polyethylenes with molecular weight of 200 to 1000 daltons; solid triglycerides; behenyl alcohol, or combinations thereof.

Chassis ingredients may be an additional structurant such as stearyl alcohol and other fatty alcohols; hydrogenated castor wax (e.g., Castorwax MP80, Castor Wax, etc.); hydrocarbon waxes include paraffin wax, beeswax, carnauba, candelilla, spermaceti wax, ozokerite, ceresin, baysberry, synthetic waxes such as Fisher-Tropsch waxes, and microcrystalline wax; polyethylenes with molecular weight of 200 to 1000 daltons; and solid triglycerides; behenyl alcohol, or combinations thereof; non-volatile organic fluids such as mineral oil, PPG-14 butyl ether, isopropyl myristate, petrolatum, butyl stearate, cetyl octanoate, butyl myristate, myristyl myristate, 02-15 alkylbenzoate (e.g., Finsolv.TM.), octyldodecanol, isostearyl isostearate, octododecyl benzoate, isostearyl lactate, isostearyl palmitate or isobutyl stearate; clay mineral powders such as talc, mica, sericite, silica, magnesium silicate, synthetic fluorphlogopite, calcium silicate, aluminum silicate, bentonite and montomorillonite; pearl pigments such as alumina, barium sulfate, calcium secondary phosphate, calcium carbonate, titanium oxide, finely divided titanium oxide, zirconium oxide, zinc oxide, hydroxy apatite, iron oxide, iron titrate, ultramarine blue, Prussian blue, chromium oxide, chromium hydroxide, cobalt oxide, cobalt titanate, titanium oxide coated mica; organic powders such as polyester, polyethylene, polystyrene, methyl methacrylate resin, cellulose, 12-nylon, 6-nylon, styrene-acrylic acid copolymers, poly propylene, vinyl chloride polymer, tetrafluoroethylene polymer, boron nitride, fish scale guanine, laked tar color dyes, laked natural color dyes; and combinations thereof.

Volatile silicone solvents suitable for use in the compositions include, but are not limited to, solvent such as Cyclomethicone D-5; GE 7207 and GE 7158 (commercially available from General Electric Co.); Dow Corning 344; Dow Corning 345; Dow Corning 200; and DC 1184 (commercially available from Dow Corning Corp.); and SWS-03314 (commercially available from SWS Silicones).

The gellant material may comprise saturated or unsaturated, substituted or unsubstituted, fatty alcohols or mixtures of fatty alcohols having from about 20 to about 60 carbons atoms, alternatively from about 20 to about 40 carbon atoms. In some embodiments, the gallant materials comprise combinations of the fatty alcohols. In some embodimens, the fatty alcohol gellants are may be saturated, unsubstituted monohydric alcohols or combinations thereof, which have a melting point of at less than about 110° C., alternatively from about 60° to about 1 10° C., alternatively between about 100° C. and 110° C.

Specific examples of fatty alcohol gellants for use in the antiperspirant products that are commercially available include, but are not limited to, Unilin® 425, Unilin® 350, Unilin® 550 and Unilin® 700 (supplied by Petrolite).

A suitable buffering agent may be alkaline, acidic or neutral. The buffer may be used in the composition or product for maintaining the desired pH. Suitable buffering agents include, for example, hydrochloric acid, sodium hydroxide, potassium hydroxide, and combinations thereof.

Non-limiting examples of suitable residue masking materials for use in the antiperspirant products include butyl stearate, diisopropyl adipate, petrolatum, nonvolatile silicones, octyldodecanol, phenyl trimethicone, isopropyl myristate, C12-15 ethanol benzoates and PPG-14 Butyl Ether.

The nature, amount and type of ingredients does not warrant a more detailed description here, which in any case would not be exhaustive, the skilled person being able to select them on the basis of its general knowledge and according to intended form.

The invention will now be described by way of the following non limiting examples.

### Example 1: Characterization of the Drying Time of a Solution-Based Fragrance Composition onto Disposable 3 Ply Masks after Application of Different Fragranced Solutions Containing Varying Amount of Ethanol

Three fragranced solutions were tested containing a constant amount of limonene (as a model fragrance) and a varying amount of ethanol and water and their respective dry time after two sprays on the inner face of a polypropylene 3 ply disposable mask was evaluated gravimetrically. It was found that a solution with 99.9% provided a suitable drying time of 5 minutes, thereby providing a composition acceptable for self-application onto the mask by the end user. The masks used here were of the “Curius” brand, manufactured by Fenghsuin Huasheng Electronics Co. Ltd, Guandgond, China, purchased in Switzerland in April 2020.

TABLE 1
Composition of the three fragranced solutions tested containing
a constant amount of limonene (fragrance) and a varying amount
of ethanol and water and their respective dry time after two
sprays on the inner face of a polypropylene 3 ply disposable
mask. The lower the ethanol content, the slower the drying.

| | Ethanol | Water | Limonene | Dry time |
|---|---|---|---|---|
| Sol 1 | 99.9% | / | 0.1% | 5 min |
| Sol 2 | 85.9% | 14% | 0.1% | 8 min |
| Sol 3 | 75.9% | 24% | 0.1% | 11 min |

### Example 2: Evaluation of the Microstructure of a Disposable Face Mask Upon Application of a Fast-Drying Fragrance Composition

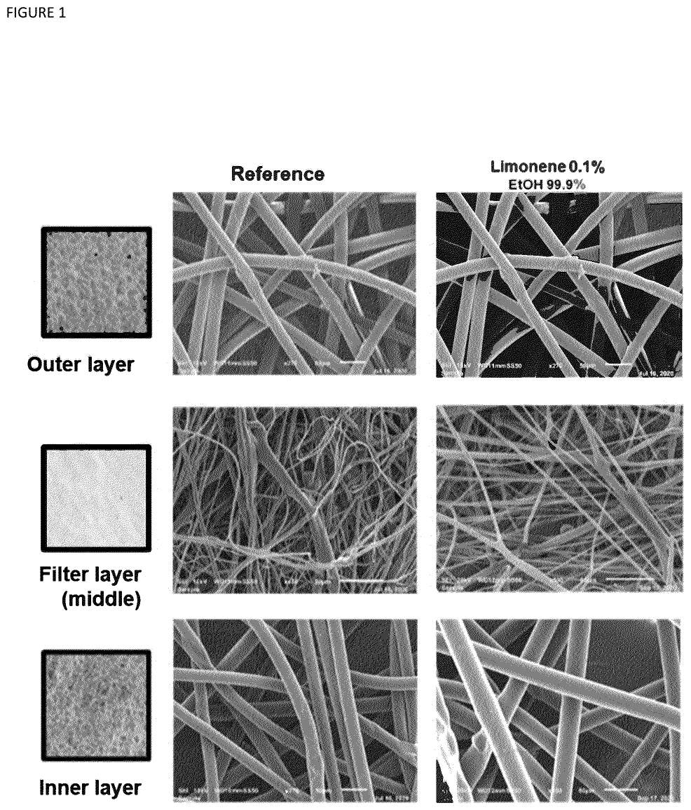

A fast-drying model fragrance composition was prepared as described in Example 1, case Solution 1, detailed in Table 1, and sprayed onto a face mask and left to dry as per Example 1. An additional mask was used “as such” as reference without any treatment. The masks where sputter-coated with a layer of gold (Denton Desk V sputter coater) for 90 seconds and scanning electron microscopy (SEM) was performed using a JSM-6010LA instrument (JEOL, Japan) in high-vacuum mode at an acceleration voltage of 15 keV. FIG. 1 demonstrates that this fragrance composition, containing 0.1%, did not affect the microstructure of any of the three layers of the mask and indeed no change was observed with respect to the fiber size, shape and roughness for the mask treated with the composition, as compared to the neat reference mask.

### Example 3: Wetting Analysis of a Disposable Face Mask Upon Application of a Fast-Drying Fragrance Composition

To evaluate whether treatment of face masks with the compositions developed in this invention affects the water-repellent properties of face masks, wetting experiments were performed using image analysis of water drops deposited onto masks treated and non-treated with the composition. A Krüss drop shape analyzer Instrument (DSA, Krüss, Germany) was used in the sessile drop configuration. The surface properties were compared by measurement of the advancing contact angle of a pure water drop on the different layers of the three-ply disposable mask in (polypropylene) before and after application of two sprays on the inner face of the mask of different fragranced solutions containing 0.1% w/w of limonene, with different amount of ethanol, as per table 1 presented in Example 1. The term ‘pure water’ refers to laboratory purpose deionized water.

Each value is an average of 10 measurements performed on the same piece of mask. The measurement of the contact angle on one layer were repeated three time on different piece of mask for reproducibility. No significant change was observed regardless of the type of solutions applied. All the contact angles measured remain far above 90° and vary only insignificantly from the reference mask, indicating that no deterioration of surface properties occurred upon treatment with the fragranced composition, even for the composition ‘Sol 1’ formulated to provide the fastest drying time according to Example 1.

### Example 4: Characterization of Disposable Three-Ply Disposable Masks after Application Different Complex Fragrance Compositions

Different complex fragrance compositions were sprayed onto three-ply masks and compared to a simple limonene/ethanol composition. The composition of the three fragranced solutions tested containing a constant amount of ethanol and fragrance of different nature (model: limonene vs. complexe fragrances) is summarized in Table 2. The compositions were applied from a spray bottle in the form of ten individual sprays, respectively five sprays on the inner face and five sprays on the outer face of a polypropylene three-ply disposable mask, were applied. These ten sprays mimic a daily usage of the sprays (two sprays every 2 h so ten sprays per day (10 h)).

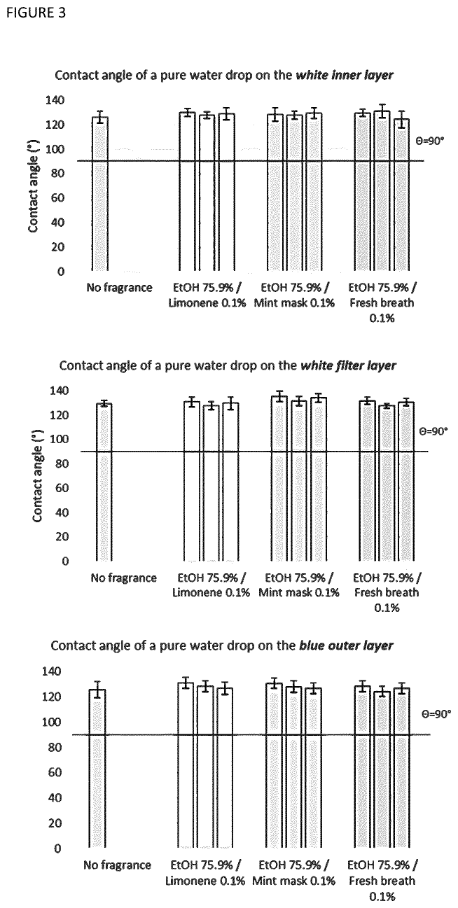

FIG. 3 shows the resulting contact angle values of a water drop, demonstrating the effect of these spray treatments on the water-repellent properties of a disposable face mask (polypropylene) before and after application of ten sprays, respectively 5 on the inner face and 5 on the outer face of the mask of different fragranced solutions containing different fragrances with a constant amount of ethanol. These ten sprays mimic a daily usage of the sprays (two sprays every 2 h so ten sprays per day (10 h)). Each value is an average of 10 measurements performed on the same piece of mask. The measurement of the contact angle on one layer were repeated three time on different piece of mask for reproducibility. No significant change was observed regardless of the fragrance used. All the contact angle measured remain above 90° and close to the reference value meaning that no loss of surface properties was identified.

TABLE 2
Composition of the three fragranced solutions tested containing
a constant amount of ethanol and fragrance of different nature
(model: limonene vs. complex fragrances) and their respective
dry time after ten sprays, respectively five sprays on the inner
face and five sprays on the outer face of a polypropylene three-
ply disposable mask. These ten sprays mimic a daily usage of the
sprays (two sprays every 2 h so ten sprays per day (10 h)).

| | Ethanol | Water | Fragrance |
|---|---|---|---|
| Sol 1 | 75.9% | 24% | Limonene 0.1% |
| Sol 2 | 75.9% | 24% | Mint mask 0.1% |
| Sol 3 | 75.9% | 24% | Fresh breath nat derived 0.1% |

### Example 5: Characterization of Two Different Textile Cloth Masks after Application of High Dosage of a “Complex” Fragranced Composition

TABLE 3
Composition of the fragranced solution tested containing a constant
amount of ethanol and a complex fragrance and its respective
dry time after ten sprays, respectively five sprays on the inner
face and five sprays on the outer face of two different type
of cloth masks. These ten sprays mimic a daily usage of the
sprays (two sprays every 2 h so ten sprays per day (10 h)).
Dry time is identical for the two types of cloth masks.

| Mask | Ethanol | Water | Fragrance | Dry time |
|---|---|---|---|---|
| Enerplex | 75.9% | 24% | Mint mask 0.1% | 60 min |
| Safe Mate | 75.9% | 24% | Mint mask 0.1% | 60 min |

### Example 6: Roll-on Device and Fragrance Composition for Application onto Face Masks

A fast-drying fragrance composition was formulated according to Table 4 and filled into a roll-on device at such compositional features and with correct roll-on sphere dimensions that the composition could be applied in a single stroke onto the lower seam of a disposable surgical face mask.

Providing the fragrance composition and roll-on stick dimensions as described in this example allows to deposit a minimum amount of fluid composition onto the mask, thereby minimizing the driving time, vet providing a satisfactory fragrance load onto the mask.

TABLE 4
Composition for use with a roll-on applicator device.
Roll-on emulsion composition

| Ingredient | Amount (wt %) |
|---|---|
| Water (Part A) | 10 |
| Hydroxyethylcellulose1) (Part A) | 1 |
| Ethanol 95% (Part B) | 90 |
| 1,2-Propylene Glycol (Part B) | 5 |
| PEG-40 Hydrogenated castor oil3) (Part C) | 4 |

1)Natrosol ® 250 H; trademark and origin: Ashland
2) Irgasan ® DP 300; trademark and origin: BASF
3)Cremophor ® RH 40; trademark and origin: BASF

Part A is prepared by sprinkling the hydroxyethylcellulose onto the water whilst rapidly stirring. Stirring is continued until the hydroxyethylcellulose is entirely swollen to form a gel. Then, Part B is poured little by little into Part A while continuing stirring until the mixture is homogeneous and part C is added.

The roll-on device consisted of a stainless steel sphere with a diameter of 10 mm; to provide the single-stroke application limited to the seam of the mask, the diameter of the sphere should be between 5 and 15 mm. Due to the soft, deformable nature of the face mask textile layers, this diameter range ensures a sufficient covering of the lower seam of the mask with the fast-drying fragrance composition while not wetting the active filtering area of the face mask (where the ‘active filtering area’ is understood to be the area on the face mask located between the lower, upper, left and right side seams, and the seams are understood to be the edge portions of the mask wherein the separate textile layers are mounted to each other).

### Example 7: Solid Compositions for Deposition with a Solid Stick Applicator onto a Face Mask

### Solid Composition 1:

| Ingredient | Amount (wt %) |
|---|---|
| Stearic acid (Part A) | 5.05 |
| 1,2-propylene glycol (Part A) | 41.87 |
| Sodium hydroxide 20% aqueous solution (Part A) | 4.24 |
| Water (Part A) | 28-30 |
| Tetrasodium EDTA¹⁾ (Part A) | 0.10 |
| Ceteareth-25 ²⁾ (Part A) | 1.52 |
| PPG-3 Myristyl ether ³⁾ (Part A) | 1.52 |
| 1,2-propylene glycol (Part B) | 15.14 |
| Fragrance composition | Complete to 100 |

Edeta ® B Power; trademark and origin: BASF
Cremophor ® A25; trademark and origin: BASF
Tegosoft ® APM; trademark and origin: Evonik
Irgasan ® DP 300; trademark and origin: BASF

All the components of Part A are weighted and heated up to 70-75° C. Ceteareth-25 is added once the other Part A ingredients are mixed and heated. Once the Ceteareth-25 is dissolved, the Stearic Acid is added. Part B is prepared by dissolving the Triclosan in 1,2 Propylene Glycol. Water which has evaporated is added. Slowly under mixing, Part B is poured into part A. The final mixture was then filled into the stick holders at a temperature of about 70° C. and left to cool down and solidify.

Another solid Composition 2 was prepared as follows using beeswax:

| Ingredient | Amount (wt %) |
|---|---|
| 1,2-propylene glycol | 82.0 |
| Beeswax¹⁾ | 7.0 |
| Sodium stearate | 8.0 |
| Fragrance composition | 3.0 |

¹⁾Beeswax yellow, supplied by ECSA Centonze, Switzerland

All the components except for the fragrance composition were mixed at room temperature in powder form and heated up to 70-75° C. (additional tests were done wherein the components were first heated and then mixed, with identical results). The fragrance composition was then added and after additional mixing with an impeller mixer for 3 minutes. The mixture was then filled into the stick holders at a temperature of about 70° C. and left to cool down and solidify.

For the above examples for solid compositions, typical batch sizes at the laboratory scale were 200 grams; larger batch sizes were prepared, in which case the mixing time was extended beyond 3 minutes to up to 8 minutes. For larger batch sizes, the mixing time should be adapted correspondingly. Homogeneity of the mixtures was evaluated visually to determine the correct mixing time.

The solid composition 2 (beeswax) is mechanically softer at a temperature 23 degC compared to solid composition 1. This example shows a way to prepare solid compositions according to the invention with different mechanical characteristics, which may be adapted to specific scenarios of use. For example, solid compositions applied to masks at cold outside temperature during winter may be formulated to remain soft even at temperatures below 10 degC, while solid compositions to be used in warm climates may be formulated to remain hard even at temperatures above 30 degC.

1. A fast-drying perfume composition comprising at least 70% of one or more fast evaporating solvents and less than 5% of one or more active ingredients.

2. The perfume composition of claim 1, wherein the fast evaporating solvent has vapor pressure of 100 Pa or more at a temperature of 25° C.

3. The perfume composition of claim 1, wherein the solvent is selected from the group consisting of ethanol, ethyl butyrate, ethyl acetate, isopropanol, 1-propanol, 1-pentanol, aromatic or aliphatic aldehydes with six or fewer C atoms, or fatty alcohols with six or fewer C atoms, 3-methoxy-3-methyl-1-butanol, amyl acetate, 1-pentanol, 2-methyl-1-butanol, 2-pentanol, 3-methyl-2-butanol, isoamyl alcohol, butyl acetate, butyl methyl ether, coconut alkanes, coco-caprylate/caprate, ethyl lactate, ethylene glycol mono n-propyl ether, 1-propanol, propylene glycol monoethyl ether, propylene glycol monomethyl ether acetate, propylene glycol monopropyl ether, lactones with a molecular weight below 80 g/mol, propiolactones with a molecular weight below 80 g/mol, cyclotrisiloxane, cyclotetrasiloxane, cimethicone, octamethyltrisiloxane, and hexamethyldisiloxane.

4. The perfume composition of claim 1, further comprising one or more of the following: water, a thickening agent and an emulsifying agent.

5. The perfume composition of claim 1, wherein the active ingredient comprises one or more of the following: perfuming ingredient, cooling agent, antimicrobial agent, antiviral agent, and malodour counteracting agent.

6. The perfume composition of claim 1, wherein the composition comprises core shell microcapsules, a polymeric carrier matrix and/or spray-dried particles.

7. A solid composition for applying a fragrance to a face mask, comprising the perfume composition of claim 1, and one or more of the following: a polymer, a fatty alcohol, a fatty acid, or salts thereof.

8. A consumer product incorporating the perfume composition of claim 1.

9. A device for applying the perfume composition of claim 1 to a face mask.

10. The device of claim 9, wherein the device is a spray bottle device or a solid stick dispenser device.

11. The device of claim 9, wherein the device further comprises a matrix material suitable for stabilizing the perfume composition during storage, to deliver the perfume composition onto the mask using the applicator, and to release the fragrance during a certain time following application to the mask.

12. A method to apply a fast-drying perfume composition to a face mask, the method comprising applying the perfume composition of claim 1 to the mask.

13. A method to manufacture a device for fragrancing a face mask, the method comprising forming the solid composition of claim 7 by preparing, blending, mixing, and heating the components, filling said solid composition into a dispenser device, and leaving the composition to solidify by letting it cool down below its temperature of solidification.

14. A consumer product incorporating the solid composition of claim 7.

15. A device for applying the solid composition of claim 7 to a face mask.

16. The device of claim 15, wherein the device is a solid stick dispenser device.

17. The device of claim 15, wherein the device further comprises a matrix material suitable for stabilizing the solid composition during storage, to deliver the solid composition onto the mask using the applicator, and to release the fragrance during a certain time following application to the mask.

18. A method to apply a fast-drying perfume composition to a face mask, the method comprising applying the solid composition of claim 7 to the mask.
